(19) United States
(12) Patent Application Publication
(10) Pub. No.: US 2023/0033224 A1
(43) Pub. Date: Feb. 2, 2023

(54) CICLESONIDE FOR AMELIORATING QUALITY OF LIFE (QOL) IN EQUINES

(71) Applicant: Boehringer Ingelheim Vetmedica GmbH, Ingelheim am Rhein (DE)

(72) Inventors: Balazs ALBRECHT, Sprendlingen (DE); Marcella VON SALIS-SOGLIO, Bad Kreuznach (DE); Saskia KLEY, Appenheim (DE)

(21) Appl. No.: 17/758,427

(22) PCT Filed: Jan. 6, 2021
(86) PCT No.: PCT/EP2021/050113

§ 371 Date: Jul. 6, 2022

(30) Foreign Application Priority Data
Jan. 10, 2020 (EP) 20151233.2

(51) Int. Cl. A61K 31/58; A61K 9/00; A61P 43/00

(52) U.S. Cl. CPC A61K 31/58; A61K 9/008; A61P 43/00

(57) ABSTRACT

The invention relates to ciclesonide or a pharmaceutically acceptable salt thereof or a composition comprising ciclesonide or a pharmaceutically acceptable salt thereof for the use in a method of ameliorating quality of life (QOL) in equines, preferably horses.

### TECHNICAL FIELD

The invention relates to ciclesonide for the use in a method of ameliorating quality of life (QOL) in equines, preferably horses.

### BACKGROUND INFORMATION

The quality of the horse's life is defined by their overall physical and mental wellbeing. The increased availability and effectiveness of veterinary procedures and medicine has resulted in increased life span of the horse; strengthening the bond between horse and owner. Longer life makes it increasingly likely that the owner will need to consider the quality of that prolonged life of their equine companion.

Assessing patients' quality of life (QOL) is a core part of clinical decision making. Various methodologies for assessing patients' QOL have been developed in human medicine and small animal veterinary disciplines. In contrast, the lack of aids for QOL assessment in equine veterinary practice leaves practitioners reliant on subjective assessments of QOL, which may be prone to avoidable errors.

Methods to assess QOL in equines have been suggested by Parker R A and Yeates J W, Equine Vet J. 2012 March; 44(2):244-9. doi: 10.1111/j.2042-3306.2011.00411.x. Epub 2011 Jul. 18.

In addition to husbandry issues such as care, exercise, and nutrition, that affects horses' QOL, there is a high need for medication, which ameliorates QOL in equines.

The problem underlying the present invention resides in providing a medication for the improvement of QOL in equines.

The International patent application WO2014/096115 A1 suggests ciclesonide for the use in a method of treating an airway disease in equines.

Surprisingly, it has been found that ciclesonide can be used in a method of ameliorating QOL in equines.

### SHORT SUMMARY OF THE INVENTION

The invention relates to ciclesonide or a pharmaceutically acceptable salt thereof or a composition comprising ciclesonide or a pharmaceutically acceptable salt thereof for the use in a method of ameliorating quality of life (QOL) in equines, preferably horses.

The amelioration of the QOL in equines can be measured by an assessment, which can be performed for example by the owner or the veterinarian or by an animal care taker or by any other person, preferably by the owner. The owners of the horses are asked to assess the quality of life of their horses over a certain period of time, e.g. several days, such as 10, 14 or 21 days, compared to a reference time point, e.g. day 0. For assessment of QOL the owner is asked to consider certain QOL parameters such as attitude, energy level, human interactions, general behavior using a rating scheme. This assessment preferably relates to a certain period of time such as for example the last 24 hours in comparison to a previous point in time for example day 0. One possible rating scheme is to ask whether QOL has improved, stayed the same or worsened compared to day 0. See the table below:

| Parameter | Assessment |
|---|---|
| Quality of Life | Improved compared to day 0 |
| | Same compared to day 0 |
| | Worsened compared to day 0 |

Another possible rating scheme is the assessment on a scale of 1 to 10 or the like. Further, the aspects attitude, energy level, human interactions, general behavior can be assessed and rated individually as well. Further or other parameters relating to QOL may be assessed as well.

An outcome of such an owner assessment for QOL amelioration in equines is shown in FIG. 1. A percentage of 60.19% of owners of horses treated with ciclosenide vs. 32.73% of owners of horses treated with placebo reported after five days of treatment that the quality of life of their horse already improved compared to Day 0. After 10(±1) days of treatment, 69.33% of the owners of the ciclosenide-treated horses vs. 43.4% of the owners of placebo-treated horses acknowledged an improvement in their horse's quality of life compared to Day 0.

In a specific embodiment of the present invention the amelioration rate of the quality of life (QOL) in equines is at least 40%, at least 50%, at least 60% after five days of treatment compared to day 0.

In a specific embodiment of the present invention the amelioration rate of the quality of life (QOL) in equines is at least 45%, at least 50%, at least 60%, at least 65%, at least 68% after ten days of treatment compared to day 0.

### SHORT DESCRIPTION OF THE DRAWINGS

FIG. 1 shows the results of the owner assessment of change in QOL on Day 5 and Day 10 compared to Day 0 from 109 treated as well as 111 placebo horses.

### DETAILED DESCRIPTION OF THE INVENTION

Before describing the various aspects of the present invention it shall be noted that as used herein and in the appended claims, the singular forms “a”, “an”, and “the” include plural reference unless the context clearly dictates otherwise. Thus, for example, reference to “a preparation” includes a plurality of such preparations reference to the “carrier” is a reference to one or more carriers and equivalents thereof known to those skilled in the art, and so forth. Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was omitted from the description. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention. Terms not specifically defined herein should be given the meanings that would be given to them by one of skill in the art in light of the disclosure and the context. As used in the specification, however, unless specified to the contrary, the following terms have the meaning indicated and the following conventions are adhered to.

The term “amelioration or improvement of quality of life (QOL) in equines” as used herein before or herein below refers to a therapeutic method involving the administration of a medicament to an equine, in particular ciclesonide, whereby the poor general health condition of the equine treated with said medicament is improved. This improvement of the general health condition may be accompanied by the treatment of a specific disease or disorder, such as a respiratory disease, in particular equine asthma, by the same or a different medicament. A poor general health condition can be due to age and thus aged or geriatric equines are one specific target population according to the present invention.

The term “ciclesonide” ((1 ip,16a)-16,17-[[(R)-Cyclohexylmethylene]bis(oxy)]-11-hydroxy-21-(2-methyl-1-oxopropoxy)pregna-1,4-diene-3,20-dione, C32H44O7, Mᵣ=540.7 g/mol) is well known in the art and means/describes a glucocorticoid used to treat asthma and allergic rhinitis in humans. It is marketed for application in humans under the brand name Alvesco™ for asthma and Omnaris™/Omniair™ for hay fever in the US and Canada. Ciclesonide is a prodrug. It is transformed into the active metabolite C21-C21-desisobutyrylciclesonide (=desciclesonide) via hydrolysis by intracellular esterases in the lung. Ciclesonide is a non-halogenated glucocorticoid, which predominantly exists in its form as R-Enantiomer.

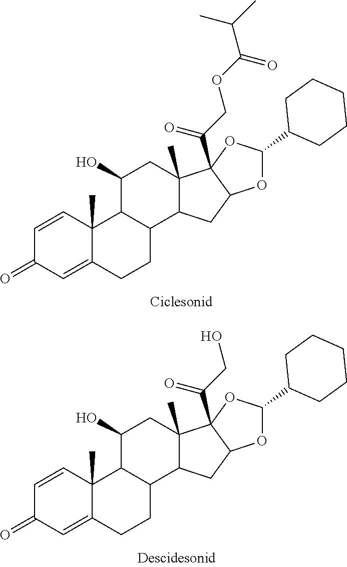 [SMILES: CC(C)C(=O)OCC(=O)[C@@]12O[C@H](C3CCCCC3)OC1CC1C3CCC4=CC(=O)C=C[C@]4(C)C3[C@@H](O)C[C@@]12C ; C[C@]12C=CC(=O)C=C1CCC1C2[C@@H](O)C[C@@]2(C)C1CC1O[C@@H](C3CCCCC3)O[C@]12C(=O)CO]

As used herein the term “prodrug” refers to (i) an inactive form of a drug that exerts its effects after metabolic processes within the body converting it to a usable or active form, or (ii) a substance that gives rise to a pharmacologically active metabolite, although not itself active (i.e. an inactive precursor).

The terms “prodrug” or “prodrug derivative” mean a covalently-bonded derivative, carrier or precursor of the parent compound or active drug substance which undergoes at least some biotransformation prior to exhibiting its pharmacological effect(s). Such prodrugs either have metabolically cleavable or otherwise convertible groups and are rapidly transformed in vivo to yield the parent compound, for example, by hydrolysis in blood or by activation via oxidation as in case of thioether groups. Most common prodrugs include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can usually be readily prepared from the parent compounds using methods known in the art. The term “equine” means of or belonging to the family Equidae, which includes the horses, asses, and zebras, preferably horses. In addition, the term “equine” encompasses also hybrids of members of the family Equidae (e.g. mules, hinnies, etc.)

The term “equine” means of or belonging to the family Equidae, which includes the horses, asses, and zebras, preferably horses. In addition, the term “equine” encompasses also hybrids of members of the family Equidae (e.g. mules, hinnies, etc.)

The term “patient” or “subject” embraces mammals such as primates including humans. The term “patient” or “subject” as used herein relates specifically to equines, preferably aged horses older than 15 years, in particular geriatric horses older than 18, 19 or 20 years, most preferred horses between 18 and 30 years that suffer from reduced QOL in particular from reduced attitude, energy levels and social interactions with human beings or other equines.

The term “aged horses” as used herein means horses older than 15 years.

The term “geriatric horses” as used herein means horses older than 18, 19 or 20 years, especially horses between 18 and 30 years.

The term “improvement rate” or “amelioration rate” means the percentage of patients/equines with improved QOL as assessed by their owners at a certain point in time (e.g. day 5 or day 10 of ciclesonide treatment) in comparison to a previous reference point in time (e.g. day 0).

The term “pharmaceutically acceptable derivative thereof” means but is not limited to pharmaceutically acceptable salts, derivatives, metabolites or pro-drugs of a drug. Derivatives as used herein include but are not limited to, any hydrate forms, solvates, isomers, enantiomers, racemates, racemic conglomerate and the like of the compound of choice. Suitable pharmaceutically acceptable salts are well known in the art and may be formed with an inorganic or organic acid, such as hydrochloric acid, hydrobromic acid, sulfuric acid, phosphoric acid, nitric acid, acetic acid, glycolic acid, lactic acid, pyruvic acid, malo-nic acid, succinic acid, glutaric acid, fumaric acid, malic acid, mandelic acid, tartaric acid, citric acid, ascorbic acid, palmitic acid, maleic acid, hydroxymaleic acid, benzoic acid, hy-droxybenzoic acid, phenylacetic acid, cinnamic acid, salicylic acid, methanesulfonic acid, benzenesulfonic acid and toluenesulfonic acid.

The dosage regimen for the treatment of a horse using ciclesonide or a composition comprising ciclesonide (as an active compound) according to the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

A physician or veterinarian can determine and prescribe the effective amount of the drug required to prevent, counter, or arrest the progress of the disorder. In the context of the present invention the term “dose” means the delivered dose “ex inhaler”. Ex inhaler comprises for example a pressurized metered dose inhaler (pMDI) or an aqueous/ethanolic droplet inhaler. A specific form of an aqueous/ethanolic droplet inhaler is for example the Respimat® inhaler or another inhalation device using the Respimat® technology. The concentration of ciclesonide contained in the solution in the inhalation device ranges preferably from 0.7 to 3.1% m/V.

The systemic dose is determined by measuring the blood levels of the prodrug (ciclesonide) and the activated metabolite (desisobutyryl-ciclesonide) in case systemic exposure is relevant in the horse. A high systemic dose results in higher side effects for example reduction of cortisol serum levels.

According to a specific aspect of the present invention said ciclesonide or said pharmaceutically acceptable salt thereof or said composition comprising ciclesonide or said pharmaceutically acceptable salt thereof is inhalable/(in the form of) an inhalant. In another aspect of the present invention the ciclesonide or the pharmaceutically acceptable salt thereof or the composition comprising ciclesonide or the pharmaceutically acceptable salt thereof is in a liquid formulation, preferably an ethanolic formulation, which can be aerosolized to facilitate its inhalation. In a further aspect of the present invention the liquid formulation is partially ethanolic and partially aqueous. In a further aspect of the present invention the liquid formulation comprises one or more of the solvents: water, ethanol, hydro fluoroal-kane(s) such as HFA 227 and HFA 134a, hydro fluoroolefin(s) such as HFO-1234ze, and optionally additional excipients. HFA is an abbreviation for hydrofluoroalkane and HFO is an abbreviation for hydrofluoroolefin.

In a preferred aspect of the present invention the solvent of the liquid formulation comprises/consists of a mixture of >85% V/V ethanol and <15% V/V water, such as for example 10%) V/V aqueous and 90% V/V ethanol. In another preferred aspect of the present invention the solvent of the liquid formulation comprises a mixture of ethanol and water, whereby the proportion of ethanol is in the range of 85-100% V/V, preferably 90-95% V/V. Preferably the proportion of ethanol is 90% V/V ethanol. In a specific aspect of the present invention the formulation (inhalation solution) of ciclesonide is as follows:

TABLE 1

| Ingredient | Content |
|---|---|
| Ciclesonide | 0.7-3.1 g/100 mL |
| Hydrochloric acid | ad [H⁺] = 10^{-3.5} to 10⁻⁵ mol/L |
| 90% V/V ethanol/water | ad 100 mL |

wherein the concentration of hydrogen ions [H] can be measured, for example, by potentiometric titration. The −log 10[H+] of this formulation is preferably in the range of 4.0 to 4.6.

A further aspect of the present invention is the application of the liquid formulation using an inhalation device, such as the Respimat® inhaler or another inhalation device using the Respimat® aerosol-generating technology. The Respimat® inhaler is disclosed for example in WO 97/12687, which is hereby incorporated by reference. This inhaler can advantageously be used to produce the inhalable aerosols/inhalants according to the invention.

The invention further concerns a method of ameliorating QOL in equines comprising administering a therapeutically effective amount of ciclesonide or a pharmaceutically acceptable salt thereof or a composition comprising ciclesonide or a pharmaceutically acceptable salt thereof to an equine patient, preferably a horse, in need thereof.

The following items are specific aspects of the present invention:

(a) Ciclesonide or a pharmaceutically acceptable salt thereof or a composition comprising ciclesonide or a pharmaceutically acceptable salt thereof according to the invention for use in a method of ameliorating the attitude, energy level, social interactions with human beings or other equines and/or general behavior in equines, preferably horses.
(b) Ciclesonide or a pharmaceutically acceptable salt thereof or a composition comprising ciclesonide or a pharmaceutically acceptable salt thereof according to the invention for use in a method of ameliorating QOL in equines, preferably horses; whereby said equines are not suffering from any airway diseases.
(c) Ciclesonide or a pharmaceutically acceptable salt thereof or a composition comprising ciclesonide or a pharmaceutically acceptable salt thereof according to the invention for use in a method of ameliorating QOL in equines, preferably horses; whereby said equines are aged or geriatric equines.
(d) Ciclesonide or a pharmaceutically acceptable salt thereof or a composition comprising ciclesonide or a pharmaceutically acceptable salt thereof according to the invention for use in a method of ameliorating QOL in equines, preferably horses; whereby the amelioration rate is at least 40%, at least 50%, or at least 60%, and/or whereby the amelioration rate is at least 40%, at least 50%, or at least 60% after five days of treatment compared to day 0.
(e) Ciclesonide or a pharmaceutically acceptable salt thereof or a composition comprising ciclesonide or a pharmaceutically acceptable salt thereof according to the invention for use in a method of ameliorating QOL in equines, preferably horses; whereby the amelioration rate is at least 45%, at least 50%, at least 60%, at least 65%, or at least 68%, and/or whereby the amelioration rate is at least 45%, at least 50%, at least 60%, at least 65%, or at least 68% after ten days of treatment compared to day 0.
(f) Ciclesonide or a pharmaceutically acceptable salt thereof or a composition comprising ciclesonide or a pharmaceutically acceptable salt thereof according to the invention for use in a method of ameliorating QOL in equines, preferably horses; whereby said ciclesonide or said pharmaceutically acceptable salt thereof or said composition comprising ciclesonide or said pharmaceutically acceptable salt thereof is inhalable.
(g) Ciclesonide or a pharmaceutically acceptable salt thereof or a composition comprising ciclesonide or a pharmaceutically acceptable salt thereof according to the invention for use in a method of ameliorating QOL in equines, preferably horses, whereby said ciclesonide or said pharmaceutically acceptable salt thereof or said composition comprising ciclesonide or said pharmaceutically acceptable salt thereof is in a liquid formulation, preferably comprising one or more of the solvents water, ethanol, hydrofluoroalkane(s) such as HFA 227 and HFA 134a, hydrofluoroolefin(s) such as HFO1234ze, and optionally additional excipients.
(h) Ciclesonide or a pharmaceutically acceptable salt thereof or a composition comprising ciclesonide or a pharmaceutically acceptable salt thereof according to the invention for use in a method of ameliorating QOL in equines, preferably horses, whereby said ciclesonide or said pharmaceutically acceptable salt thereof or said composition comprising ciclesonide or said pharmaceutically acceptable salt thereof is in a liquid formulation, preferably the solvent in said liquid formulation is partially aqueous and partially ethanolic, most preferably the solvent in said liquid formulation consists of a mixture of ≥85% V/V ethanol and ≤15% V/V water such as for example 10% V/V water and 90% V/V ethanol.
(i) Ciclesonide or a pharmaceutically acceptable salt thereof or a composition comprising ciclesonide or a pharmaceutically acceptable salt thereof according to the invention for use in a method of ameliorating QOL in equines, preferably horses, whereby said ciclesonide or said pharmaceutically acceptable salt thereof or said composition comprising ciclesonide or said pharmaceutically acceptable salt thereof is administered via an (equine) inhaler device.
(j) Ciclesonide or a pharmaceutically acceptable salt thereof or a composition comprising ciclesonide or a pharmaceutically acceptable salt thereof according to item (g) for use in a method of ameliorating QOL in equines, preferably horses, whereby said inhaler device comprises:
a. a pressurized metered dose inhaler or an aqueous/ethanolic droplet inhaler such as the Respimat® inhaler or another inhalation device using the Respimat® aerosol-generating technology and
b. an adapter for equine use.
   (k) Ciclesonide or a pharmaceutically acceptable salt thereof or a composition comprising ciclesonide or a pharmaceutically acceptable salt thereof according to to the invention for use in a method of ameliorating QOL in equines, preferably horses, whereby said ciclesonide or said pharmaceutically acceptable salt thereof or said composition comprising ciclesonide or said pharmaceutically acceptable salt thereof is a partially ethanolic formulation and is administered via an (equine) inhaler device.
   (l) Ciclesonide or a pharmaceutically acceptable salt thereof or a composition comprising ciclesonide or a pharmaceutically acceptable salt thereof according to the invention for use in a method of ameliorating QOL in equines, preferably horses, whereby said ciclesonide or said pharmaceutically acceptable salt thereof or said composition comprising ciclesonide or said pharmaceutically acceptable salt thereof is administered at a dose of at least 900 μg ex inhaler, at least 1800 μg ex inhaler, at least 2400 μg ex inhaler, or at least 2700 μg ex inhaler, preferably at least 2700 μg ex inhaler.
   (m) Ciclesonide or a pharmaceutically acceptable salt thereof or a composition comprising ciclesonide or a pharmaceutically acceptable salt thereof according to the invention for use in a method of ameliorating QOL in equines, preferably horses, whereby said ciclesonide or said pharmaceutically acceptable salt thereof or said composition comprising ciclesonide or said pharmaceutically acceptable salt thereof is administered at a dose of 100 μg to 5000 μg ex inhaler, 450 μg to 2700 μs ex inhaler, 900 μg to 2400 μs ex inhaler, 900 μg to 2700 μs ex inhaler, preferably at a dose of 900 μg to 2700 μg ex inhaler.
   (n) Ciclesonide or a pharmaceutically acceptable salt thereof or a composition comprising ciclesonide or a pharmaceutically acceptable salt thereof according to the invention for use in a method of ameliorating QOL in equines, preferably horses, whereby said ciclesonide or said pharmaceutically acceptable salt thereof or said composition comprising ciclesonide or said pharmaceutically acceptable salt thereof is administered with less than 20 actuations per dose, preferably 12, 11, 10, 9, 8, 7, 6, 5, 4, 3, 2, or 1 actuation per dose, most preferably said ciclesonide or said pharmaceutically acceptable salt thereof or said composition comprising ciclesonide or said pharmaceutically acceptable salt thereof is administered using 8 or fewer actuations per dose.
   (o) Ciclesonide or a pharmaceutically acceptable salt thereof or a composition comprising ciclesonide or a pharmaceutically acceptable salt thereof according to the invention for use in a method of ameliorating QOL in equines, preferably horses, whereby said ciclesonide or said pharmaceutically acceptable salt thereof or said composition comprising ciclesonide or said pharmaceutically acceptable salt thereof is administered in 1 to 3 doses per day, preferably 1 to 2 doses per day.
   (p) Ciclesonide or a pharmaceutically acceptable salt thereof or a composition comprising ciclesonide or a pharmaceutically acceptable salt thereof according to the invention for use in a method of ameliorating QOL in equines, preferably horses, whereby said ciclesonide or said pharmaceutically acceptable salt thereof or said composition comprising ciclesonide or said pharmaceutically acceptable salt thereof is administered over an extended time period of at least 1 week, at least 2, 3, 4, 5, 6, 7, 8, 9, or 10 weeks or more.
   (q) A method of assessing the amelioration rate of the quality of life (QOL) of an equine treated with ciclesonide including the following steps:
(i) assessing the QOL of an equine and/or certain QOL parameters such as attitude, and/or energy level, and/or social interactions with human beings or other equines and/or general behavior, preferably by its owner, at a first point in time before ciclesonide treatment (e.g. day 0 of ciclesonide treatment)
(ii) assessing the QOL of an equine and/or certain QOL parameters such as attitude, and/or energy level, and/or social interactions with human beings or other equines and/or general behavior, preferably by its owner, at a second later point in time during or at the end of ciclesonide treatment (e.g. day 5 or day 10 of ciclesonide treatment)
(iii) comparing the QOL improvement ratings of the second later point in time to the previous first (reference) point in time.

### EXAMPLES

The following examples serve to further illustrate the present invention; but the same should not be construed as a limitation of the scope of the invention disclosed herein.

### Example 1

Study Design

The study is conducted as a multicenter prospective, randomized, double-blinded, placebo-controlled clinical study with parallel group design according to Good Clinical Practice (GCP) guidelines with client-owned horses.

Horses are examined before (Day 0) and at the end of treatment with the study drug (Day 10±1) by the study investigators. Physical examination, efficacy and QOL assessment, as well as blood sample collection is performed during the visits. The owners of included horses are also contacted by telephone on Day 5.

Animals and Inclusion/Exclusion Criteria

Client-owned, adult horses of any sex and breed without and with moderate to severe clinical signs of equine asthma are eligible for inclusion, provided informed owner consent is obtained and the horses comply with all predetermined inclusion and exclusion criteria.

The administration of routine treatments (e.g., vaccinations, antiparasitic drugs) is permitted. Throughout the duration of the study, animals are kept in their usual environment and their management, exercise and dietary regimes remained the same.

Treatment

Horses are randomly assigned, at a ratio of 1:1, to receive either inhaled ciclesonide solution or an inhaled placebo solution. The randomization scheme is designed such that an equal number of ciclesonide and placebo treated horses can be included at each site, thus minimizing any site-associated treatment bias.

Inhaled treatment is administered using a newly developed inhaler based on Respimat® technology (called thereafter EquiHaler®), consisting of a nostril adapter attached to a soft mist inhaler (SMI) core unit. During breathing, deflection of a respiration indicator (“breath indicator”) located in the chamber wall of the nostril adapter facilitated easy identification of inspiration and expiration. Placebo is administered using the EquiHaler® with cartridges containing the excipients but lacking ciclesonide.

For the first five days of the study period, horses are administered 8 actuations of either ciclesonide (1 actuation delivers 343 μg ciclesonide) or placebo solution twice daily, approximately 12 hours apart. For the subsequent five days of the study, horses are administered 12 actuations once daily, either in the morning or the evening. This selected dosing regimen is based on data derived from prior dose titration experiments and studies aimed at identifying appropriate dose administration frequencies, conducted on environmentally challenged asthma-susceptible research horses.

Assessment

The owners of the horses in the trial are asked to assess the quality of life of their horses compared to Day 0.

The owner is asked to consider attitude, energy level, human interactions, general behaviour, etc. of the last 24 hours before the visit using the scheme of the following table:

TABLE 2

| Parameter | Assessment |
|---|---|
| Quality of Life | Improved compared to day 0 |
| | Same compared to day 0 |
| | Worsened compared to day 0 |

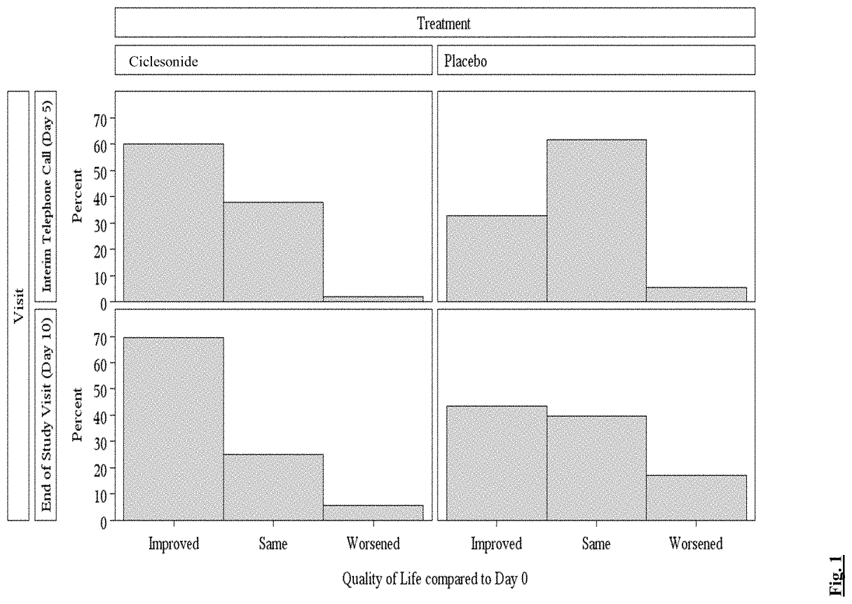

The results of this owner assessment are shown in FIG. 1.

A percentage of 60.19% of owners of horses treated with ciclosenide vs. 32.73% of owners of horses treated with placebo reported after five days of treatment that the quality of life of their horse already improved compared to Day 0. After 10(±1) days of treatment, 69.33% of the owners of the ciclosenide-treated horses vs. 43.4% of the owners of placebo-treated horses acknowledged an improvement in their horse's quality of life compared to Day 0.

1. Ciclesonide or a pharmaceutically acceptable salt thereof or a composition comprising ciclesonide or a pharmaceutically acceptable salt thereof for the use in a method of ameliorating quality of life (QOL) in equines, preferably horses.

2. Ciclesonide or a pharmaceutically acceptable salt thereof or a composition comprising ciclesonide or a pharmaceutically acceptable salt thereof according to claim 1 for use in a method of ameliorating the attitude, and/or energy level, and/or social interactions with human beings or other equines and/or general behavior in equines, preferably horses.

3. Ciclesonide or a pharmaceutically acceptable salt thereof or a composition comprising ciclesonide or a pharmaceutically acceptable salt thereof according to claim 1 or 2 for use in a method of ameliorating QOL in equines, preferably horses; whereby said equines are not suffering from any airway diseases.

4. Ciclesonide or a pharmaceutically acceptable salt thereof or a composition comprising ciclesonide or a pharmaceutically acceptable salt thereof according to claim claims 1 to 3 for use in a method of ameliorating QOL in equines, preferably horses; whereby said equines are aged or geriatric equines.

5. Ciclesonide or a pharmaceutically acceptable salt thereof or a composition comprising ciclesonide or a pharmaceutically acceptable salt thereof according to claims 1 to 4 for use in a method of ameliorating QOL in equines, preferably horses; whereby the amelioration rate is at least 40%, at least 45%, at least 50%, at least 60%, at least 65%, or at least 68%, preferably the amelioration rate is at least 40%, at least 50%, or at least 60% after five days of treatment compared to day 0 or the amelioration rate is at least 45%, at least 50%, at least 60%, at least 65%, or at least 68% after ten days of treatment compared to day 0.

6. Ciclesonide or a pharmaceutically acceptable salt thereof or a composition comprising ciclesonide or a pharmaceutically acceptable salt thereof according to claims 1 to 5 for use in a method of ameliorating QOL in equines, preferably horses, whereby said ciclesonide or said pharmaceutically acceptable salt thereof or said composition comprising ciclesonide or said pharmaceutically acceptable salt thereof is inhalable, or whereby said ciclesonide or said pharmaceutically acceptable salt thereof or said composition comprising ciclesonide or said pharmaceutically acceptable salt thereof is in a liquid formulation, preferably comprising one or more of the solvents water, ethanol, hydrofluoroalkane(s) such as HFA 227 and HFA 134a, hydrofluoroolefin(s) such as HFO1234ze, and optionally additional excipients.

7. Ciclesonide or a pharmaceutically acceptable salt thereof or a composition comprising ciclesonide or a pharmaceutically acceptable salt thereof according to claims 1 to 6 for use in a method of ameliorating QOL in equines, preferably horses, whereby said ciclesonide or said pharmaceutically acceptable salt thereof or said composition comprising ciclesonide or said pharmaceutically acceptable salt thereof is in a liquid formulation, preferably the solvent in said liquid formulation is partially aqueous and partially ethanolic, most preferably the solvent in said liquid formulation consists of a mixture of ≥85% V/V ethanol and ≤15% V/V water such as for example 10% V/V water and 90% V/V ethanol.

8. Ciclesonide or a pharmaceutically acceptable salt thereof or a composition comprising ciclesonide or a pharmaceutically acceptable salt thereof according to claims 1 to 7 for use in a method of ameliorating QOL in equines, preferably horses, whereby said ciclesonide or said pharmaceutically acceptable salt thereof or said composition comprising ciclesonide or said pharmaceutically acceptable salt thereof is administered via an (equine) inhaler device.

9. Ciclesonide or a pharmaceutically acceptable salt thereof or a composition comprising ciclesonide or a pharmaceutically acceptable salt thereof according to claim 8 for use in a method of ameliorating QOL in equines, preferably horses, whereby said inhaler device comprises:
a. a pressurized metered dose inhaler or an aqueous/ethanolic droplet inhaler such as the Respimat® inhaler or another inhalation device using the Respimat® aerosol-generating technology and
b. an adapter for equine use.

10. Ciclesonide or a pharmaceutically acceptable salt thereof or a composition comprising ciclesonide or a pharmaceutically acceptable salt thereof according to claims 1 to 9 for use in a method of ameliorating QOL in equines, preferably horses, whereby said ciclesonide or said pharmaceutically acceptable salt thereof or said composition comprising ciclesonide or said pharmaceutically acceptable salt thereof is a partially ethanolic formulation and is administered via an (equine) inhaler device.

11. Ciclesonide or a pharmaceutically acceptable salt thereof or a composition comprising ciclesonide or a pharmaceutically acceptable salt thereof according to claims 1 to 10 for use in a method of ameliorating QOL in equines, preferably horses, whereby said ciclesonide or said pharmaceutically acceptable salt thereof or said composition comprising ciclesonide or said pharmaceutically acceptable salt thereof is administered at a dose of at least 900 μg ex inhaler, at least 1800 μg ex inhaler, at least 2400 μg ex inhaler, or at least 2700 μg ex inhaler, preferably at least 2700 μg ex inhaler.

12. Ciclesonide or a pharmaceutically acceptable salt thereof or a composition comprising ciclesonide or a pharmaceutically acceptable salt thereof according to claims 1 to 11 for use in a method of ameliorating QOL in equines, preferably horses, whereby said ciclesonide or said pharmaceutically acceptable salt thereof or said composition comprising ciclesonide or said pharmaceutically acceptable salt thereof is administered at a dose of 100 μg to 5000 μg ex inhaler, 450 μg to 2700 μs ex inhaler, 900 μg to 2400 μs ex inhaler, 900 μg to 2700 μs ex inhaler, preferably at a dose of 900 μg to 2700 μg ex inhaler.

13. Ciclesonide or a pharmaceutically acceptable salt thereof or a composition comprising ciclesonide or a pharmaceutically acceptable salt thereof according to claims 1 to 12 for use in a method of ameliorating QOL in equines, preferably horses, whereby said ciclesonide or said pharmaceutically acceptable salt thereof or said composition comprising ciclesonide or said pharmaceutically acceptable salt thereof is administered with less than 20 actuations per dose, preferably 12, 11, 10, 9, 8, 7, 6, 5, 4, 3, 2, or 1 actuation per dose, most preferably said ciclesonide or said pharmaceutically acceptable salt thereof or said composition comprising ciclesonide or said pharmaceutically acceptable salt thereof is administered using 8 or fewer actuations per dose.

14. Ciclesonide or a pharmaceutically acceptable salt thereof or a composition comprising ciclesonide or a pharmaceutically acceptable salt thereof according to claims 1 to 13 for use in a method of ameliorating QOL in equines, preferably horses, whereby said ciclesonide or said pharmaceutically acceptable salt thereof or said composition comprising ciclesonide or said pharmaceutically acceptable salt thereof is administered in 1 to 3 doses per day, preferably 1 to 2 doses per day.

15. Ciclesonide or a pharmaceutically acceptable salt thereof or a composition comprising ciclesonide or a pharmaceutically acceptable salt thereof according to claims 1 to 14 for use in a method of ameliorating QOL in equines, preferably horses, whereby said ciclesonide or said pharmaceutically acceptable salt thereof or said composition comprising ciclesonide or said pharmaceutically acceptable salt thereof is administered over an extended time period of at least 1 week, at least 2, 3, 4, 5, 6, 7, 8, 9, or 10 weeks or more.

16. A method of assessing the amelioration rate of the quality of life (QOL) of an equine treated with ciclesonide including the following steps:
(i) assessing the QOL of an equine and/or certain QOL parameters such as attitude, and/or energy level, and/or social interactions with human beings or other equines and/or general behavior, preferably by its owner, at a first point in time before ciclesonide treatment (e.g. day 0 of ciclesonide treatment)
(ii) assessing the QOL of an equine and/or certain QOL parameters such as attitude, and/or energy level, and/or social interactions with human beings or other equines and/or general behavior, preferably by its owner, at a second later point in time during or at the end of ciclesonide treatment (e.g. day 5 or day 10 of ciclesonide treatment)
(iii) comparing the QOL improvement ratings of the second later point in time to the previous first (reference) point in time.
